(19) United States
(12) Patent Application Publication
(10) Pub. No.: US 2024/0216460 A1
(43) Pub. Date: Jul. 4, 2024

(54) SALVIA PLEBIA AND SCUTELLARIAE RADIX COMPOUND ACNE CREAM, PREPARATION METHOD THEREFOR AND APPLICATION THEREOF

(71) Applicant: SOOCHOW UNIVERSITY, Suzhou (CN)

(72) Inventors: Jialu YAO, Suzhou (CN); Hong YANG, Suzhou (CN); Yuanyuan LI, Suzhou (CN); Liang CHEN, Suzhou (CN); Jiangyun LIU, Suzhou (CN); Ming LIU, Suzhou (CN)

(21) Appl. No.: 18/012,321

(22) PCT Filed: Nov. 23, 2020
(86) PCT No.: PCT/CN2020/130963

§ 371 Date: Jul. 19, 2023

(51) Int. Cl. A61K 36/539; A61K 9/06; A61K 36/537

(52) U.S. Cl. CPC A61K 36/539; A61K 9/06; A61K 36/537; A61K 2236/333

(57) ABSTRACT

A Salvia plebia and Scutellariae radix compound acne cream, a preparation method therefor and an application thereof. Stearic acid, vaseline, lanolin, glycerol monostearate and cetyl alcohol are heated and mixed to obtain an oil phase; glycerol and triethanolamine are fusion-mixed to obtain a water phase; and the water phase, the oil phase, a Scutellariae radix extract and a Salvia plebia extract are mixed, water is added to specified volume and the mixture is mixed uniformly to obtain the Salvia plebia and Scutellariae radix compound acne cream. The problems of large toxic and side effects, long treatment period and drug resistance of pathogenic bacteria of existing acne-treating drugs are solved. The Chinese medicine-based cream is prepared from a novel external preparation cream, has strong skin adhesion and good biocompatibility, and can effectively facilitate drugs to penetrate stratum corneum, reach an action site and exert a curative effect. The drug bioavailability is enhanced, the drug dosage is reduced, the curative effect is guaranteed, irritation caused by excessive local drug concentration can be avoided, and the use is convenient and safe.

### TECHNICAL FIELD

The invention belongs to the technology of traditional Chinese medicine preparations, and particularly relates to a Salvia plebia and Scutellariae radix compound acne cream as well as a preparation method and application thereof traditional Chinese medicine.

### BACKGROUND TECHNOLOGY

Acne is mainly caused by Propionibacterium acnes, but also caused by Malassezia furfur, Staphylococcus epidermidis and the like. Propionibacterium acnes (Propionibacterium acnes), which are widely present in pilosebaceous glands, are conditionally pathogenic bacteria that mainly cause endogenous infections. The inflammatory response triggered by Propionibacterium acnes is the pathogenesis of acne. Propionibacterium acnes secretes lipase, which breaks down triglycerides in sebum, and the free fatty acids formed stimulate the antimicrobial polypeptides (H β D1 and 2, antimicrobial peptides and granulysin) to activate downstream inflammatory responses. Propionibacterium acnes can directly act on Toll-like receptors (TLR 2 and TLR 4) of keratinocytes and inflammatory cells to generate inflammatory chemokines, neutrophils and macrophages are gathered into hair follicles through IL-8, and then the promoted inflammatory reaction causes the destruction of hair follicle walls and the rapid proliferation of the macrophages, so that a large amount of IL-8 and IL-12 are further released. TLR-mediated cytokines can also activate AP-1, can produce metalloproteases, and cause tissue destruction and scar formation. TLRs can also stimulate antimicrobial polypeptide secretion. Propionibacterium acnes promotes hyperkeratosis of hair follicles by producing integrins and mesosilk-related proteins. The Propionibacterium acnes can also form a biomembrane by secreting components such as polysaccharide and the like to wrap the Propionibacterium acnes so as to promote the attachment of the Propionibacterium acnes in hair follicles, and the biomembrane can not only further promote hyperkeratosis, but also increase the drug resistance of the Propionibacterium acnes to antibiotics.

### Technical Problems

At present, the acne treatment method mainly comprises external medicines, chemotherapy, physical therapy, oral traditional Chinese medicines and the like. The external medicines comprise tretinoin, benzoyl peroxide, vitamins and the like, and although the external medicines are used as first-line medicines and even first-choice medicines for treating acne, the external medicines often cause skin irritation problems such as local redness, desquamation, tightness, burning sensation and the like, and easily cause the consequences of cross drug resistance, increase of skin photosensitivity, pigmentation, slight skin atrophy at the applied part and the like, so that the clinical application of the external medicines is limited. In addition, chemotherapy such as tartaric acid, and photodynamic therapy (PDT), although nontoxic, odorless and permeable, have high cost and high energy consumption, and may cause facial problems such as erythema after treatment. Oral administration of traditional Chinese medicine may cause hepatotoxicity, and the acceptance of patients is generally low due to the problems of processing, decoction, drug administration frequency, long treatment time and the like. The traditional Chinese medicine has a long history of treating acne, has certain characteristics in the aspect of treating acne, has small adverse reaction, and has better explanation on the action mechanism of treating acne with the traditional Chinese medicine along with the continuous and deep research on the traditional Chinese medicine in recent years. The traditional Chinese medicine considers that: the generation of acne is closely related to the damp-heat in lung and stomach, and the normal transportation and transformation can be affected by the condition that spleen and stomach are damaged due to improper diet; if people drink wine too much, people often eat greasy fish-smell products, which can help dampness and heat to cause the exuberant cortex and greasy and shiny skin, so that vital energy and blood of people become stagnated and are emitted to the skin to generate pustules. For a long time, blood stasis and vital energy stagnation form cysts, nodules and the like. Most of the traditional Chinese medicine external preparations are hard to permeate keratinized skin, and have poor permeability, low absorption rate and poor curative effect.

### DISCLOSURE OF INVENTION

The invention discloses a Salvia plebia and Scutellariae radix compound acne cream and a preparation method thereof, which overcomes the problems of great toxic and side effects, long treatment period, drug resistance of pathogenic bacteria and the like of the existing acne treatment drugs. The prepared new external preparation cream is made into traditional Chinese medicine cream, which has strong adhesion to the skin, high biocompatibility, can effectively promote the penetration of the stratum corneum, reach the deep action site, exert the efficacy, not only improve the bioavailability of the drug, reduce the drug dose, ensure the efficacy, but also avoid the irritation problems caused by the local high drug concentration, and is convenient and safe to use. Therefore, the research on a new type of traditional Chinese medicine preparation, Salvia plebia and Scutellariae radix compound acne cream, is of great significance.

The invention adopts the following technical scheme: Salvia plebia and Scutellariae radix compound acne cream comprises Scutellaria-extract, Salvia plebia extract, and adjuvants; the adjuvants are water phase adjuvants and oil phase adjuvants.

In the invention, the water phase adjuvant is a mixture of glycerol, water and triethanolamine; the oil phase adjuvant is mixture of stearic acid, vaseline, lanoline, glyceryl monostearate, and cetyl alcohol.

In the invention, in 100 mL of Salvia plebia and Scutellariae radix compound acne cream, the dosage of Scutellaria-extract is 2-3 g; the dosage of the Salvia plebia extract is 2-3 g. Preferably, the dosage of the Salvia plebia extract is equal to that of the Scutellaria-extract, and the Salvia plebia extract and the Scutellaria-extract are both 2.5 g.

Further, 100 mL of the Salvia plebia and Scutellariae radix compound acne cream contains the following adjuvants: 1 g of stearic acid, 10 g of vaseline, 8 g of lanolin, 4 g of glyceryl monostearate, 8 g of hexadecanol, 13 g of glycerol, 6 g of triethanolamine and the balance is water.

In the invention, Salvia plebia is extracted by ethanol and concentrated to obtain Salvia plebia extract; preferably, the dosage of the ethanol is 12 times of the weight of the Salvia plebia; the extraction temperature is 60° C. and is the external heating temperature; after extraction, the mixture is distilled under reduced pressure at 60° C. to obtain Salvia plebia extract. Scutellariae radix is extracted with ethanol, and concentrated to obtain Scutellaria-extract, preferably, the dosage of the ethanol is 12 times of the weight of of Scutellariae radix; the extraction temperature is 80° C., and is the external heating temperature; and after extraction, the mixture is distilled under reduced pressure at 60° C., and concentrated to 1/50- 1/100 of the original volume to obtain the Scutellaria-extract. Preferably, the ethanol is 75 vol % ethanol water solution (v/v).

In the invention, the Salvia plebia and Scutellariae radix compound acne cream comprises Scutellaria-extract, Salvia plebia extract and adjuvants, and is prepared by heating and mixing stearic acid, vaseline, lanoline, glyceryl monostearate and hexadecanol to obtain an oil phase; melting and mixing glycerol and triethanolamine to obtain a water phase; mixing the water phase, the oil phase, the Scutellaria-extract, the Salvia plebia extract and water to obtain the Salvia plebia and Scutellariae radix compound acne cream. Preferably, the water phase is added into the oil phase, then the Scutellaria-extract and the Salvia plebia extract are added, and finally water is added to obtain the Salvia plebia and Scutellariae radix compound acne cream.

The invention discloses application of the Salvia plebia and Scutellariae radix compound acne cream in preparing a medicine for treating acne. The invention also discloses an application of the compound of Salvia plebia and Scutellariae radix in preparing a medicine for treating acne, and particularly, the compound of Salvia plebia and Scutellariae radix is a compound of Salvia plebia extract and Scutellaria-extract.

### Beneficial Effects

In order to overcome the problems of the existing acne treatment medicines, the invention starts with the pharmacological action and treatment characteristics of Salvia plebia and Scutellariae radix, designs to prepare the extracts of the Salvia plebia and the Scutellariae radix into cream for acne treatment, prepares the extracts into novel external preparation traditional Chinese medicine cream, has strong adhesion to skin and high biocompatibility, can effectively promote the medicines to penetrate through the stratum corneum of the skin and reach the action part to exert curative effect, not only improves the bioavailability of the medicines, reduces the medicine dosage, ensures the curative effect, but also can avoid the irritation problem caused by overhigh local medicine concentration, and is convenient and safe to use. Therefore, the research idea of the Salvia plebia and Scutellariae radix compound acne cream is feasible, and all technical processes of related research methods can be realized under the existing process.

### DRAWINGS

FIG. 1 is an HPLC-UV chromatogram of a homoplantaginin control;

FIG. 2 is an HPLC-UV chromatogram of a test sample (Salvia plebia ethanol extract, Salvia plebia extract);

FIG. 3 is HPLC-UV chromatogram of baicalin control;

FIG. 4 is HPLC-UV chromatogram of the test sample (Scutellariae radix ethanol extract, Scutellaria-extract);

FIG. 5 shows the results of a preliminary experiment of bacteriostatic activity of an ethanol extract of Salvia plebia in different concentrations by a filter paper sheet method, (B: 95% ethanol, 0.5:0.5% ethanol extract solution of Salvia plebia, 1.25:1.25% ethanol extract solution of Salvia plebia);

FIG. 6 shows the results of a preliminary experiment of bacteriostatic activity of an ethanol extract of sage in different concentrations by a filter paper method (P: penicillin; C: cefradine; A; amikacin; S: streptomycin);

FIG. 7 shows bacteriostatic activity of Salvia plebia extract solution, Scutellariae radix ethanol extract solution and mixed extract solution in different concentrations for Bacillus subtilis by filter paper method (H16:16% Baical skullcap root extract soluble in acetone; H8:8% Baical skullcap root extract soluble in acetone; H4:4% Baical skullcap root extract soluble in acetone; H2:2% Baical skullcap root extract soluble in acetone; H1:1% Baical skullcap root extract soluble in acetone; H0.5:0.5% Baical skullcap root extract soluble in acetone; L16:16% Salvia plebia alcohol extract soluble in acetone; L8:8% Salvia plebia alcohol extract soluble in acetone; L4:4% Salvia plebia alcohol extract soluble in acetone; L2:2% Salvia plebia alcohol extract soluble in acetone; L1:1% Salvia plebia alcohol extract soluble in acetone; L0.5:0.5% Salvia plebia alcohol extract soluble in acetone; HL4:1:8% Baical skullcap root extract soluble in acetone and 2% Salvia plebia alcohol extract soluble in acetone; HL2:1:6.7% Baical skullcap root extract soluble in acetone and 3.3% Salvia plebia alcohol extract soluble in acetone; HL1:1:5% Baical skullcap root extract soluble in acetone and 5% Salvia plebia alcohol extract soluble in acetone; HL1:2:3.3% Baical skullcap root extract soluble in acetone and 6.7% Salvia plebia alcohol extract soluble in acetone; HL4:1:2% Baical skullcap root extract soluble in acetone and 8% Salvia plebia alcohol extract soluble in acetone).

FIG. 8 shows bacteriostatic activity of Salvia plebia ethanol extract, Scutellariae radix ethanol extract and mixed alcohol extract in different concentrations for Staphylococcus aureus by filter paper method (H16:16% Baical skullcap root extract soluble in acetone; H8:8% Baical skullcap root extract soluble in acetone; H4:4% Baical skullcap root extract soluble in acetone; H2:2% Baical skullcap root extract soluble in acetone; H1:1% Baical skullcap root extract soluble in acetone; H0.5:0.5% Baical skullcap root extract soluble in acetone; L16:16% Salvia plebia alcohol extract soluble in acetone; L8:8% Salvia plebia alcohol extract soluble in acetone; L4:4% Salvia plebia alcohol extract soluble in acetone; L2:2% Salvia plebia alcohol extract soluble in acetone; L1:1% Salvia plebia alcohol extract soluble in acetone; L0.5:0.5% Salvia plebia alcohol extract soluble in acetone; HL4:1:8% Baical skullcap root extract soluble in acetone and 2% Salvia plebia alcohol extract soluble in acetone; HL2:1:6.7% Baical skullcap root extract soluble in acetone and 3.3% Salvia plebia alcohol extract soluble in acetone; HL1:1:5% Baical skullcap root extract soluble in acetone and 5% Salvia plebia alcohol extract soluble in acetone; HL1:2:3.3% Baical skullcap root extract soluble in acetone and 6.7% Salvia plebia alcohol extract soluble in acetone; HL4:1:2% Baical skullcap root extract soluble in acetone and 8% Salvia plebia alcohol extract soluble in acetone).

FIG. 9 shows the centrifugation result of the Salvia plebia and Scutellariae radix compound acne cream;

FIG. 10 is a Salvia plebia and Scutellariae radix compound acne cream.

### EXAMPLES

### Materials:

Salvia plebia (Nanjing Shangyuantang Pharmaceutical Co., Ltd., batch No. 190704);
Scutellariae radix (Suzhou Tianling Chinese Herbal Medicine Co. Ltd., batch No. 130617-1);
triethanolamine (Sinopharm Chemical Reagent Co., Ltd);
Propionibacterium acnes (Institute of Microbiology, Chinese Academy of Sciences);
Staphylococcus aureus (ATCC), Staphylococcus epidermidis (ATCC), Bacillus subtilis (ATCC), Staphylococcus albus (ATCC);
glycerin (Sinopharm Chemical Reagent Co., Ltd); white petrolatum (8009-03-8);
lanolin (Suzhou Gretel Pharmaceutical Technology Co., Ltd.);
glyceryl monostearate (Suzhou Kesaien Biotechnology Co., Ltd);
hexadecanol (Suzhou Tesley Scientific Instruments Co., Ltd);
homoplantaginin control (Shanghai yuanye Bio-Technology Co., Ltd);
baicalin control (Shanghai yuanye Bio-Technology Co., Ltd);
stearic acid (Suzhou Tesley Scientific Instruments Co., Ltd);
polypeptone (Jiangsu Argon Krypton Xenon Material Technology Co., Ltd);
tryptone (Suzhou Shengnuokang Biotechnology Co., Ltd.);
yeast extract (Jiangsu Argon Krypton Xenon Material Technology Co., Ltd);
glucose (Suzhou Kesaien Biotechnology Co., Ltd);
agar 15 (Suzhou Tesley Scientific Instruments Co., Ltd).

The instruments include a rotary evaporator; an electric heating constant-temperature water bath kettle; a centrifuge; an ultra-clean bench; an electronic analytical balance; a manual pipettor; a refrigerator; high performance liquid chromatography (HPLC); C18 280 nm chromatographic column (250 mm×4.6 mm, 5 μm); a chemical workstation; a column oven; an anaerobic chamber; an ultrasonic cleaner.

All the raw materials of the invention are commercial products, and the specific operation method and the preparation method are conventional technologies. The creativity of the invention lies in that the Scutellaria-extract and the Salvia plebia extract are combined to realize the effective inhibition of Propionibacterium acnes, and it is mixed with medical adjuvants to obtain the Salvia plebia and Scutellariae radix compound acne cream.

### Example 1

The dried whole Salvia plebia was taken and extracted by using 75 vol % ethanol with the weight being 12 times of that of the dried whole Salvia plebia under the condition of hot reflux (60° C.) for 3 times, and extracted for 2 hours each time; the extractive solutions of the 3 were put together, distilled at 60° C. under reduced pressure to obtain Salvia plebia extract 1 mg/mL, and tested at room temperature.

The dried Scutellariae radix powder was taken and soaked by 75 vol % ethanol with the weight being 12 times for 2 h and extracted under reflux (80° C.) for 3 times (each time for 2 hr); the extractive solutions of the 3 were put together, distilled at 60° C. under reduced pressure to obtain Scutellaria-extract 1.01 mg/mL, and tested at room temperature.

1 g of stearic acid, 10 g of vaseline, 8 g of lanolin, 4 g of glyceryl monostearate and 8 g of hexadecanol were heated and melted on a water bath at 85° C. to obtain an oil phase for later use; 13 g of glycerol and 6 g of triethanolamine were melted and mixed at 85° C., and added into the oil phase, and the heating was removed to cool down to below 60° C., and the extract, namely 2.5 g of the Scutellaria-extract and 2.5 g of the Salvia plebia extract were added, and then water was added to a constant volume of 100 mL, and naturally cooled to obtain the Salvia plebia and Scutellariae radix and compound acne cream. The above preparation was carried out under conventional stirring.

HPLC method was used to measure the concentration of baicalin and homoplantaginin in alcohol extract.

### Preparation of the Relevant Solutions

Preparation of mixed control solution: 5 mg of baicalin and 5 mg of homoplantain as reference substances were accurately weighed and placed in a 10 mL volumetric flask, dissolved with analytical methanol under ultrasonic condition and diluted to constant volume to obtain a reference substance stock solution, and refrigerated at 4° C.

Treatment of the test solution: it was filtered through a 0.45 m filter before use.

Chromatographic Condition for Measuring Concentration of Salvia plebia

A C18 280 nm chromatographic column (250 mm×4.6 mm, 5 mu m) was selected; mobile phase: 0.1% phosphoric acid water (A)-methanol (B), gradient elution (15% B-38% B at 0-8 min, 38% B-56% B at 8-17 min, 56% B-82% B at 17-28 min, 82% B-95% B at 28-35 min); flow rate: 1 mL/min; the detection wavelength was 335 nm; column temperature: 25° C.; sample size: 10 μL.

The system applicability was as follows: the sample volume of the reference substance and the test solution was 10 mu L respectively. It was analyzed according to the chromatographic conditions, and number of theoretical plates was calculated to be all more than 8000.

### Chromatographic Condition for Measuring Concentration of Baicalin.

A C18 280 nm chromatographic column (250 mm×4.6 mm, 5 mu m) was selected; mobile phase: 0.1% methanol-water-phosphoric acid (47:53:0.2), gradient elution (15% B-38% B at 0-8 min, 38% B-56% B at 8-17 min, 56% B-82% B at 17-28 min, 82% B-95% B at 28-35 min); flow rate: 1 mL/min; the detection wavelength was 280 nm; column temperature: 25° C.; sample size: 10 μL.

The system applicability was as follows: the sample volume of the reference substance and the test solution was 10 mu L respectively. It was analyzed according to the chromatographic conditions, and number of theoretical plates was calculated to be all more than 8000.

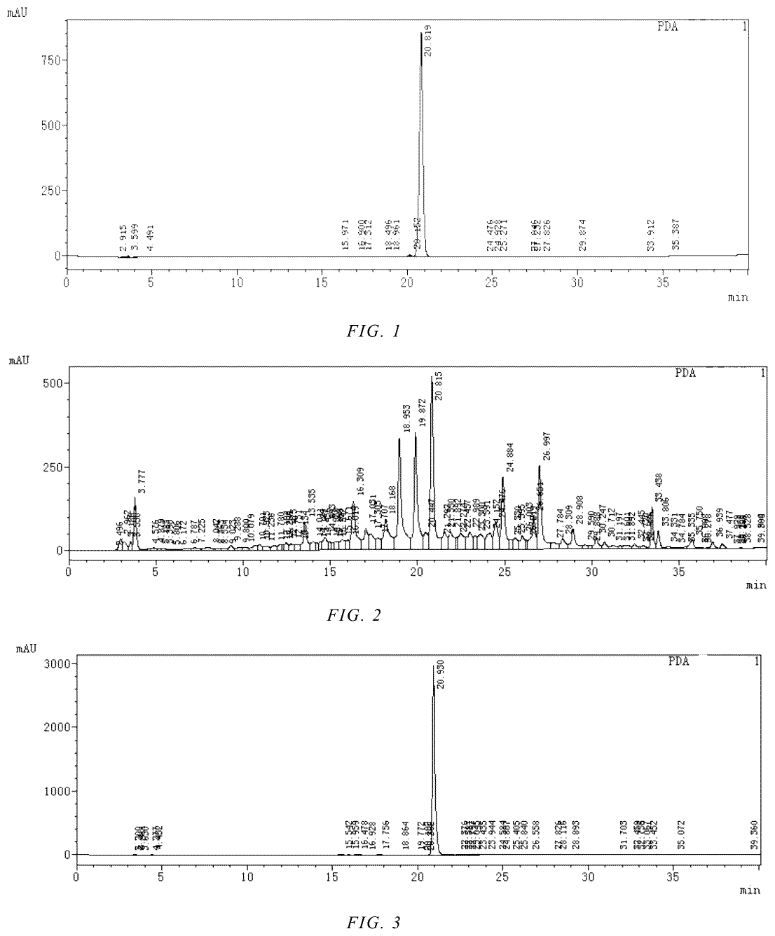

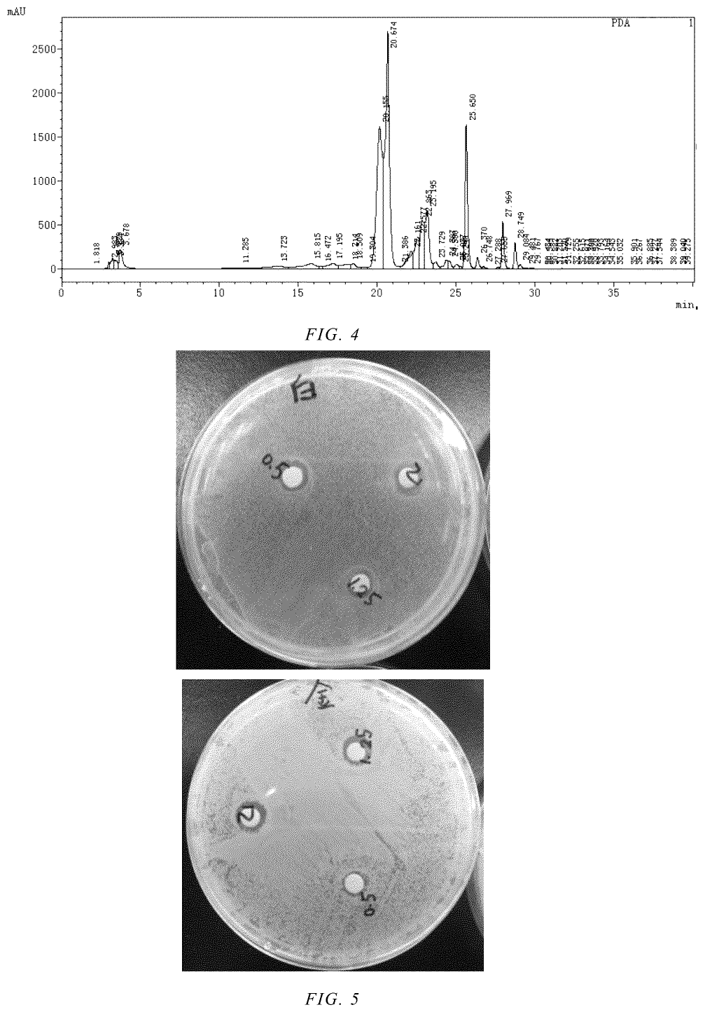

FIG. 1 is HPLC-UV chromatogram of a homoplantaginin reference substance; FIG. 2 is HPLC-UV chromatogram of the sample (Salvia plebia ethanol extract, Salvia plebia extract); FIG. 3 is HPLC-UV chromatogram of baicalin reference substance; FIG. 4 is HPLC-UV chromatogram of the sample (Scutellariae radix ethanol extract, Scutellaria-extract). In the drawings, the peak value is clear, and can indicate that the extract of the invention has corresponding components, and other values do not influence the understanding of the person skilled in the art.

Characterization of Salvia plebia and Scutellariae Radix Compound Acne Cream

### Centrifugal Experiment

10 g of cream was taken and placed in a centrifuge tube, centrifuged at 4500 r/min, rotated for 10 min to observe whether delamination occurred.

### Heat Resistance Test

The cream was taken and placed into a glass ware to be placed in a thermostat, the temperature was controlled at 60° C. for 6 h, and then it was taken out to observe whether it had delamination at room temperature.

### Cold Resistance Test

The cream was taken and placed in a glass ware, and placed in a refrigerator at −15° C., and it was taken out after 24 hr, to observe whether the product had delamination phenomenon after it returned to room temperature.

### Evaluation Standard of Appearance of Cream

Inspect the properties of the prepared cream, and score according to five grades in the following table by taking the gloss, the fineness, the sand feel and the coating performance as judgment standards.

TABLE 1
Evaluation criteria for cream properties

| Numbering | Scoring | Evaluation |
|---|---|---|
| 1 | 4-5 | It has fine and smooth appearance, gloss, no |
| | | sand feeling and easy coating |
| 2 | 3-4 | It has fine and smooth appearance, gloss, no |
| | | sand feeling and general coating |
| 3 | 2-3 | It has fine and smooth appearance, no obvious |
| | | gloss, slight sand feeling and slight greasy |
| | | feeling |
| 4 | 1-2 | It has fine and smooth appearance, no obvious |
| | | gloss, slight sand feeling and heavy greasy |
| | | feeling |
| 5 | 0-1 | Insoluble substances can be seen with naked |
| | | eyes, no obvious gloss, obvious sand feeling |
| | | and heavy greasy feeling |

### Bacteriostasis Test

### Recovery of Bacteria

Resuscitation of Propionibacterium acnes

### Recovery Culture of Vacuum Freeze-Dried Strain

1. Unsealing the ampoule: the outer surface of the ampoule was sterilized by 75% alcohol absorbent cotton, then the top end of the ampoule was heated by flame, sterile water was dropped, the heated top end of the ampoule was broken, and the broken top end of the ampoule was knocked down by tweezers.

2. Recovery culture: 0.3 mL of liquid culture medium (i.e., recommended medium formulation provided by CGMCC without agar) was absorbed by a disposable pipette (sterile), dropped into an ampoule, and shaken to dissolve it and become suspended. All the bacterial suspension was sucked and transplanted into a culture medium test tube for static culture.

### Matters of Attention

1. Before resuming cultivation of the bacterial strain, store the received bacterial strain ampoule in an environment of 6-10 degrees Celsius.

2. When resuming the cultivation of anaerobic bacteria, maintain anaerobic conditions; During the production of the culture medium, anaerobic spiral shaped test tubes need to be used and high-purity NH₃ is used to remove oxygen.

The medium formula is shown in Table 2.

TABLE 2
formulation of 0244 PYG medium

| | | |
|---|---|---|
| Polypeptone | 5.0 | g |
| Tryptone | 5.0 | g |
| yeast extract | 10.0 | g |
| glucose | 10.0 | g |
| salt solution | 40.0 | mL |
| distilled water | 960.0 | mL |
| agar 15 | 15.0 | g |
| pH 7.2 | | |
| salt solution: | | |
| CaCl₂ | 0.2 | g |
| MgSO₄•7H₂O | 0.4 | g |
| K₂HPO₄ | 1.0 | g |
| KH₂PO₄ | 1.0 | g |
| NaHCO₃ | 10.0 | g |
| NaCl | 2.0 | g |
| Deionized water | 1.0 | L |

Recovery of Staphylococcus aureus, Staphylococcus epidermidis, Bacillus subtilis, Staphylococcus albicans

The tested strain was taken out from an ultra-low temperature refrigerator at −80° C., inoculated to 3 mL of LB liquid culture medium, cultured overnight at 37° C., and transferred to a blood plate for activation. The activated bacteria were inoculated to an LB solid culture medium by zoning and marking and a single colony was selected for biochemical identification and then used for experiments; meanwhile, a single colony was picked and inoculated on an LB inclined plane, and was refrigerated and stored at 4° C. for short-term use. Before the experiment, single colonies were picked from LB plates to 3 mL of LB liquid medium, and cultured at 37° C. with shaking at a low speed until logarithmic phase.

### Preparation of the Bacterial Suspension

A ring of each strain was taken and diluted with sterile water to obtain a strain concentration of 10⁵-10⁶ cfu/mL for use.

The filter paper method was used to investigate the inhibitory ability of extracts from Salvia plebia and Scutellariae radix on pathogenic bacteria.

The bacteriostatic activity test of the Salvia plebia and Scutellariae radix alcohol extracts with different concentrations used the filter paper method to carry out a bacteriostatic zone test. The plate was treated with the bacterial suspension by the coating method. Then, the sterilized filter paper sheets were attached to the plates, respectively, and 3 filter paper sheets (repeated 3 times) were placed on the culture plate at equal intervals; the acetone was taken as a blending medium to prepare extracts with different concentrations as a solution to be tested, for example, adding acetone to 2.5 g of Salvia plebia extract to 100 mL to obtain a 2.5% Salvia plebia extract solution (Salvia plebia ethanol extract acetone solution), adding acetone to 2.5 g of Salvia plebia extract and 2.5 g of Scutellaria-extract to 100 mL to obtain a 2.5% Salvia plebia/2.5% Scutellaria-extract solution. And respectively dripping acetone, sterile water and the solution to be tested on the filter paper sheet. Aerobic culture of Staphylococcus aureus, Staphylococcus epidermidis, Bacillus subtilis and Staphylococcus albus and anaerobic culture of Propionibacterium acnes were carried out in an incubator at 37° C., and results were observed after 24 hours, particularly with the conventional technology. Taking the bacteriostatic zone as a bacteriostatic activity index, and according to the bacteriostatic zone test judgment standard:

TABLE 3
Determination criteria for inhibition zone

| | Antibacterial | Inhibition zone | |
|---|---|---|---|
| Number | effect | size (mm) | Note |
| 1 | Insensitive | D ≤ 7 mm | The inhibition |
| 2 | Low sensitive | 7 mm < D ≤ 10 mm | zone is clearly |
| 3 | Medium sensitive | 10 mm < D ≤ 15 mm | visible with |
| 4 | Highly sensitive | 15 mm < D ≤ 20 mm | regular edges and |
| 5 | Extremely sensitive | D > 20 mm | structured shapes |

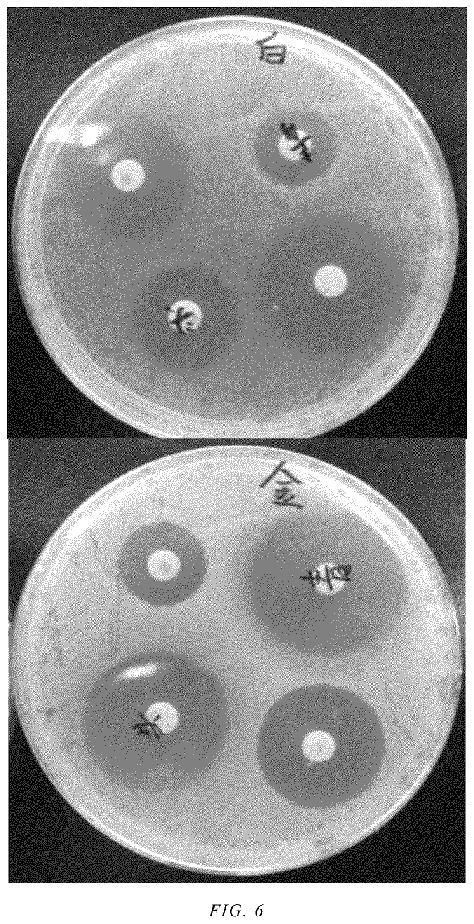

FIG. 5 shows the results of inhibiting Staphylococcus aureus and Staphylococcus albus with 0.5% Salvia plebia extract solution and 1.25% Salvia plebia extract solution, and FIG. 6 shows the results of inhibiting Staphylococcus aureus and Staphylococcus albus with penicillin, cefradine, amikacin, and streptomycin as positive control, using standard test paper. Experiments show that the Salvia plebia ethanol extract has antibacterial activity on Staphylococcus albus and Staphylococcus aureus, the antibacterial zone is obvious and clearly visible, the edges are regular, and the shapes are structured. The inference results show that the Salvia plebia ethanol extract has good bacteriostatic activity.

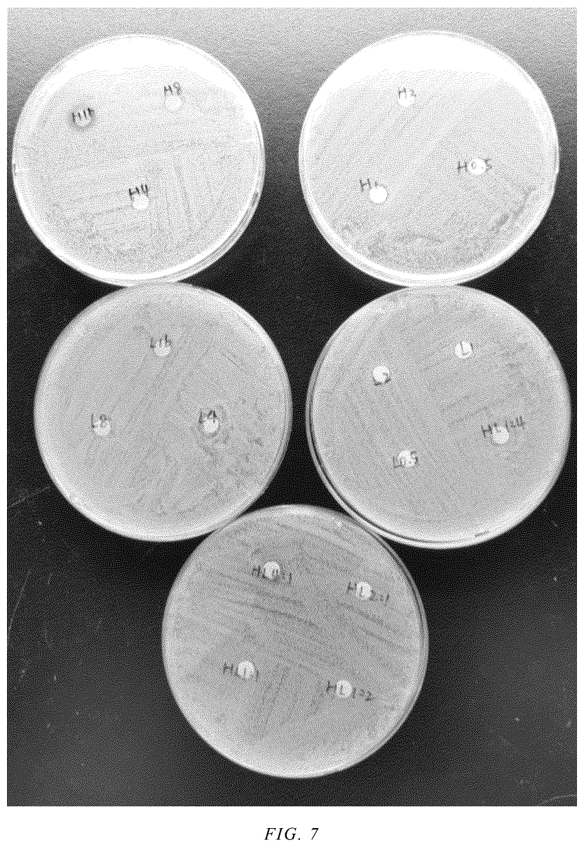

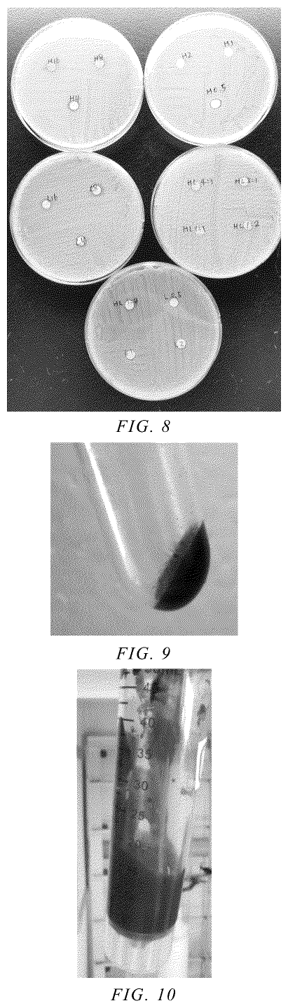

FIG. 7 shows bacteriostatic activity of Salvia plebia extract solution, Scutellariae radix ethanol extract solution and mixed extract solution of different concentrations on Bacillus subtilis by a filter paper sheet method; FIG. 8 shows bacteriostatic activity of Salvia plebia extract solution, Scutellariae radix ethanol extract solution and mixed extract solution of different concentrations on Staphylococcus aureus by using a filter paper sheet method.

It can be seen from the experiment that the concentration of Scutellariae radix alcohol extract and the alcohol extract of lichee grass have antibacterial activity, and part of the inhibition zone is obvious and clearly visible, with regular edges and regular shapes; The inhibitory effects of Bacillus, Staphylococcus epidermidis, and Staphylococcus aureus were not positively correlated; for Bacillus subtilis, Staphylococcus epidermidis, and Staphylococcus aureus, it was preliminarily concluded that the synergistic effect of Scutellariae radix and Litchi grass was not good. See Table 4, where “/” indicates that the inhibition zone is less than or equal to 6 mm, the diameter of the inhibition zone is obtained from the average of three replicates, and the difference between the three replicates is less than 0.1 mm, which can also indicate that the stability of the antibacterial effect of the drug is good.

TABLE 4
The antibacterial activity of Bacillus subtilis, Staphylococcus epidermidis,
and Staphylococcus aureus by the filter paper method of different
concentrations of Salvia Plebia extract solution, baicalein
extract solution and mixed extract solution.

| | | bacil- | Staphy- | |
|---|---|---|---|---|
| | | lus | lococcus | Staphy- |
| fungus/inhibition | | sub- | epi- | lococcus |
| zone (mm) | | tilis | dermidis | aureus |
| Baical | 16% Baical skullcap root | 9.87 | 7.50 | 8.77 |
| skullcap | extract soluble in acetone | | | |
| root | 8% Baical skullcap root | 7.16 | 7.77 | 6.92 |
| extract | extract soluble in acetone | | | |
| | 4% Baical skullcap root | 6.58 | 8.59 | 6.62 |
| | extract soluble in acetone | | | |
| | 2% Baical skullcap root | 6.77 | 7.23 | 6.46 |
| | extract soluble in acetone | | | |
| | 1% Baical skullcap root | 7.35 | / | 7.69 |
| | extract soluble in acetone | | | |
| | 0.5% Baical skullcap root | 7.35 | 8.73 | 7.69 |
| | extract soluble in acetone | | | |
| Salvia | 16% Salvia Plebia alcohol | 7.35 | 7.23 | 6.77 |
| Plebia | extract soluble in acetone | | | |
| alcohol | 8% Salvia Plebia alcohol | 7.16 | 7.23 | 7.23 |
| extract | extract soluble in acetone | | | |
| | 4% Salvia Plebia alcohol | 7.74 | 7.09 | / |
| | extract soluble in acetone | | | |
| | 2% Salvia Plebia alcohol | / | 7.09 | / |
| | extract soluble in acetone | | | |
| | 1% Salvia Plebia alcohol | / | 7.36 | / |
| | extract soluble in acetone | | | |
| Mixture | 8% Baical skullcap root | 7.94 | 7.50 | 7.69 |
| of Baical | extract soluble in acetone | | | |
| skullcap | and 2% Salvia Plebia | | | |
| root | alcohol extract soluble in | | | |
| extract | acetone | | | |
| soluble | 6.7% Baical skullcap root | / | 6.82 | 6.8 |
| in acetone | extract soluble in acetone | | | |
| and | and 3.3% Salvia Plebia | | | |
| Salvia | alcohol extract soluble in | | | |
| Plebia | acetone | | | |
| alcohol | 5% Baical skullcap root | 6.39 | / | 6.29 |
| extract | extract soluble in acetone | | | |
| soluble | and 5% Salvia Plebia | | | |
| in acetone | alcohol extract soluble in | | | |
| | acetone | | | |
| | 3.3% Baical skullcap root | / | 7.09 | / |
| | extract soluble in acetone | | | |
| | and 6.7% Salvia Plebia | | | |
| | alcohol extract soluble in | | | |
| | acetone | | | |
| | 2% Baical skullcap root | 7.35 | 7.09 | / |
| | extract soluble in acetone | | | |
| | and 8% Salvia Plebia | | | |
| | alcohol extract soluble in | | | |
| | acetone | | | |

The following experiments show that: the concentration of Scutellariae radix ethanol extract and the Salvia plebia alcohol extract have antibacterial activity, part of antibacterial zones is obvious and clearly visible, the edges are regular, and the shapes are structured; the concentration of the Scutellariae radix ethanol extract and the concentration of the Salvia plebia ethanol extract are not positively correlated with the inhibition effects of Bacillus subtilis, Staphylococcus epidermidis and Staphylococcus aureus; for Bacillus subtilis, Staphylococcus epidermidis and Staphylococcus aureus, the synergistic effect of the Scutellariae radix and the Salvia plebia is not good preliminarily. See table 4, where “/” indicates that the inhibition zone is less than or equal to 6 mm, the diameter of the inhibition zone is obtained by averaging three duplicate samples, and the difference between the three duplicate samples is less than 0.1 mm, which also indicates that the stability of the drug's bacteriostatic effect is good.

According to the existing research, the active ingredients in the Salvia plebia can inhibit the release of inflammatory factors through a TLR4/MyD88/NF-KB signal channel to play an anti-inflammatory role; the Scutellariae radix and the active ingredients thereof are used as natural products, and the bacteriostatic mechanism of the Scutellariae radix is usually realized by multiple ways of influencing the formation and permeability of bacterial cell membranes, interfering the synthesis of protein in a bacterium, inhibiting the metabolism of the bacterium, inhibiting DNA topoisomerase and the like; the synergistic mechanism of the two drugs is probably the combination of the respective bacteriostatic mechanisms of the two drugs, and further research and study on the bacteriostatic mechanism and the synergistic mechanism of the combined drugs and whether the drugs have regulation and control effects on other pathogenic links are needed. The etiology and pathogenesis of acne are complex, and include factors such as sex hormone level, keratosis of pilosebaceous canal, infection of Propionibacterium acnes, immunity and inflammation, wherein the infection of Propionibacterium acnes is an important factor for causing acne formation. Research has proved that the number of P. acnes at the rash part of the acne patient is increased, and after the treatment by using the antibacterial drug, the number of the P. acnes is reduced, which is parallel to the improvement of clinical symptoms.

The cream is used as a novel external preparation, is suitable for chronic skin lesions such as thickening of epidermis, keratinization, rhagadia and the like or early infectious skin diseases, and can promote the effectiveness of drugs only at the site of action to play a curative effect; the traditional Chinese medicine cream is a dosage form for external administration of traditional Chinese medicines, and is an excellent dosage form for treating various diseases through skin, orifices, meridians and collaterals, pathological change local parts and other parts, the medicine is uniformly distributed in an oil-water two-phase matrix and released after reaching an action part, so that the bioavailability of the medicines is improved, the medicine dosage is reduced, the curative effect is ensured, the irritation problem caused by overhigh local medicine concentration is solved, and the traditional Chinese medicine cream is convenient and safe to use.

The centrifugation result of the Salvia plebia and Scutellariae radix compound acne cream is shown in FIG. 9, the cream (shown in FIG. 10) is brown semisolid, the texture is uniform and fine, the coating property is good, no obvious granular sensation exists, and the Salvia plebia and Scutellariae radix compound acne cream meets the requirement.

### Summary

The invention takes the traditional Chinese medicines of the Salvia plebia and the Scutellariae radix as raw materials, researches and prepares the Salvia plebia and the Scutellariae radix compound acne cream, establishes a quality control method, measures the transdermal property and the bacteriostatic ability, and lays a foundation for further developing a novel efficient, low-toxic and convenient external preparation of traditional Chinese medicine compound acne. The result confirms the extraction conditions of the Salvia plebia and Scutellariae radix acne cream: the Salvia plebia is extracted for 3 times with 75% ethanol of 12 times weight under hot reflux (60° C.) for 2 hours each time. Soaking Scutellariae radix in 75% ethanol of 12 times weight for 2 hr, and extracting under reflux at 80° C. for 3 times for 2 hours each time. The ethanol extract of Scutellariae radix and the Salvia plebia have bacteriostatic activity, and have obvious and clear inhibition zone, regular edges and structured shapes. The prescription disclosed by the invention comprises 2.5 g of Scutellaria-extract, 2.5 g of Salvia plebia extract, 13 g of glycerol, 6 g of triethanolamine, 1 g of stearic acid, 10 g of vaseline, 8 g of lanolin, 4 g of glyceryl monostearate and 8 g of hexadecanol, and constant volume to 100 mL with water; the cream is brown semisolid, has uniform and fine texture, good coating property and no obvious granular sensation, and meets the medical requirements.

1. A Salvia plebia and Scutellariae radix compound acne cream, characterized in that it comprises a Scutellaria-extract, a Salvia plebia extract, and adjuvants; the adjuvants include a water phase adjuvant and an oil phase adjuvant.

2. The Salvia plebia and Scutellariae radix compound acne cream according to claim 1, characterized in that, the water phase adjuvant is a mixture of glycerol, water and triethanolamine; the oil phase adjuvant is mixture of stearic acid, vaseline, lanoline, glyceryl monostearate, and cetyl alcohol.

3. The Salvia plebia and Scutellariae radix compound acne cream according to claim 1, characterized in that, in 100 mL of the Salvia plebia and Scutellariae radix compound acne cream, an amount of the Scutellaria-extract is 2-3 g; an amount of the Salvia plebia extract is 2-3 g.

4. The Salvia plebia and Scutellariae radix compound acne cream according to claim 1, characterized in that, an amount of the Salvia plebia extract is the same as the Scutellaria-extract.

5. The Salvia plebia and Scutellariae radix compound acne cream according to claim 1, characterized in that, Salvia plebia is extracted by ethanol and concentrated to obtain the Salvia plebia extract; Scutellariae radix is extracted with ethanol, and concentrated to obtain the Scutellaria-extract.

6. The Salvia plebia and Scutellariae radix compound acne cream according to claim 5, characterized in that, an amount of the ethanol is 12 times of an amount of the Salvia plebia; an extraction temperature is 60° C. when extracting Salvia plebia with ethanol.

7. The Salvia plebia and Scutellariae radix compound acne cream according to claim 5, characterized in that, an amount of the ethanol is 12 times of an amount of Scutellariae radix; an extraction temperature is 80° C. when extracting Scutellariae radix with ethanol.

8. The Salvia plebia and Scutellariae radix compound acne cream according to claim 5, characterized in that, the ethanol is 75 vol % ethanol water solution (v/v); and after extraction, a mixture is distilled under reduced pressure at 60° C.

9. A preparation method of the Salvia plebia and Scutellariae radix compound acne cream according to claim 1 comprising: heating and mixing stearic acid, vaseline, lanoline, glyceryl monostearate and hexadecanol to obtain an oil phase; melting and mixing glycerol and triethanolamine to obtain a water phase; mixing the water phase, the oil phase, the Scutellaria-extract, the Salvia plebia extract and water to obtain the Salvia plebia and Scutellariae radix compound acne cream.

10. An application of the Salvia plebia and Scutellariae radix compound acne cream according to claim 1 in the preparation of acne treatment medicine or the application of the Litchi grass and Scutellariae radix compound in the preparation of acne treatment medicine.
